(19) United States
(12) Patent Application Publication
(10) Pub. No.: US 2026/0272872 A1
(43) Pub. Date: Sep. 17, 2026

(54) FAT COMPOSITION COMPRISING TRIBUTYRIN AND ITS USE IN CALF MILK REPLACERS

(71) Applicant: Nutreco IP Assets B.V., Boxmeer (NL)

(72) Inventors: Leonel Neto LEAL, Boxmeer (NL); Juliette Nina Roxanne WILMS, Boxmeer (NL); Gerrit Jan KLAASSEN, Boxmeer (NL)

(21) Appl. No.: 19/648,182

(22) Filed: Apr. 15, 2026

Related U.S. Application Data
(63) Continuation of application No. PCT/EP2024/079232, filed on Oct. 16, 2024.

(30) Foreign Application Priority Data
Oct. 16, 2023 (EP) 23203814.1

(51) Int. Cl. A61K 31/225; A23D 9/007; A23D 9/02; A23K 20/147; A23K 20/158; A23K 20/163; A23K 40/10; A23K 50/10; A23K 50/60; A61K 9/16; A61P 1/12; A61P 3/00

(52) U.S. Cl. CPC A61K 31/225; A23D 9/007; A23D 9/02; A23K 20/147; A23K 20/158; A23K 20/163; A23K 40/10; A23K 50/10; A23K 50/60; A61K 9/1617; A61K 9/1623; A61K 9/1658; A61P 1/12; A61P 3/00

(57) ABSTRACT

The present invention provides a fat composition comprising a) at least 0.7 wt % tributyrin; b) 25-60 wt % fats, e.g., vegetable and/or animal fats; c) 18-75 wt % protein, e.g., milk protein and optionally vegetable protein; and d) optionally, carbohydrates such as lactose, as well as the use thereof in a composition for feeding young farming mammals, in particular to improve their performance and gastrointestinal development.

### CROSS REFERENCE TO RELATED APPLICATIONS

The present application is a continuation of International Application No. PCT/EP2024/079232, filed Oct. 16, 2024, which claims priority to European Patent Application No. 23203814.1, filed Oct. 16, 2023, both of which are hereby incorporated by reference herein in their entireties.

### FIELD OF THE INVENTION

The present invention is in the field of fat compositions that may be used in various applications, in particular in liquid feeds for young farming animals, e.g., milk replacer, as well as such liquid feeds for young farming animals.

### BACKGROUND OF THE INVENTION

Young mammals such as calves rely heavily on milk in the early stages of life to provide proteins, energy and essential nutrients to grow and stay healthy. The energy from milk comes from milk sugars (lactose) and fat.

Cow's milk naturally contains a balanced fatty acid profile with all 3 categories (small-chain, medium-chain, and long-chain) of fatty acids represented.

Calf milk replacer (CMR) was historically developed to spare saleable milk. However, milk replacer is now valued for its ability to provide a convenient, safe, consistent, and cost-effective source of nutrients for pre-weaned calves. A proper calf milk replacer should have physical, chemical and nutritional characteristics comparable to milk (flowability, density, homogeneity and palatability). It has to supply sufficient energy and nutrients. Milk replacers are therefore designed to contain the ingredients needed to provide the appropriate content of protein, fat, carbohydrates, minerals and vitamins. Moreover, it should dissolve readily in water to ease feeding.

In Europe and other parts of the world, mostly vegetable oils are used in CMRs, since they are cheaper than milk fat and are a rich source of other fatty acids, such as C12:0 and C16:0. In many other parts of the world, animal fats such as lard and tallow are used as a source of fatty acids. However, short-chain fatty acids such as butyric acid (C4:0) are not present in vegetable oils or animal fats used in current CMRs, whereas caproic acid (C6:0) is only present in small amounts in coconut oil. Currently available CMRs are therefore unlikely to contain the amount of C4:0 and C6:0 required for optimal calf development and health.

Butyric and caproic acid can be supplemented in different forms in CMR, including in the form of free fatty acids, salts or esters. Free C4:0 and C6:0 are water-soluble, colorless, oily liquids. These free fatty acids are volatile, corrosive, and have a bad odour. They are therefore difficult to use in animal feed.

To ease C4:0 and C6:0 supplementation, C4:0 and C6:0 can be added in the form of solid butyrate and caproate salts or soaps (calcium, sodium, potassium, or magnesium), or esterified with glycerol to obtain butyrins or caproins. Salts have several benefits over free acids including being les odorous and easier to control in feed manufacturing operations, since they are solid and less volatile. Esters are liquid at room temperature, making them easier to supplement in CMR. Because of its wide availability and low cost, sodium butyrate is mainly used as a source of dietary C4:0 in commercial CMRs. However, inclusion levels of sodium butyrate are limited by its bad odour.

Trials with butyrate salt supplementation in CMRs have shown promising effects; however, the amount of butyrate salt that can be added to milk replacer is limited by its foul smell.

Tributyrin may be added to CMRs instead of butyrate salts. Tributyrins do not have a bad odour, but they have an astringent taste that may repulse calves and hence depress feed intake.

Tributyrin has been added up to a level of 0.6 wt % of dry matter in CMR (Murayama et al. 2023. J. Dairy Sci. 106:4599-4607). The authors observed an increased total dry matter intake during postweaning in calves fed milk replacer with tributyrin. Calves fed milk replacer with 0.6 wt % of dry matter content tributyrin also had greater body weight during the weaning and postweaning period compared with calves that had not been fed tributyrin.

It is an objective of the present invention to provide a fat concentrate suitable for use in CMR and a CMR comprising such fat concentrate that increases pre-weaning body weight gain, weaning body weight gain, post-weaning body weight gain, pre-weaning average daily gain, weaning average daily gain, post-weaning average daily gain, milk replacer intake, starter feed intake, dry matter intake, decreases energy conversion ratio, reduces diarrhoea incidence and/or severity and/or respiratory disease incidence and/or severity in a young farming mammal such as a calf, and/or that improves rumen development and/or ileal development in a young farming mammal such as a calf, and/or that improves fat digestibility and/or insulin sensitivity in a young farming mammal, such as a calf.

Additionally, or alternatively, it is an object of the present invention to provide a fat concentrate comprising tributyrin and optionally tricaproin that can optionally be combined with commonly used and/or commercially available fat concentrates for CMR in a desired ratio to provide a CMR with a desired tributyrin and optionally tricaproin content. In particular, the presently presented technology enables higher inclusion levels of tributyrin in CMR.

### SUMMARY OF THE INVENTION

The present disclosure provides a composition comprising: a) at least 0.7 wt % tributyrin; b) 25-60 wt % fats; c) 18-75 wt % protein, e.g., milk protein and optionally vegetable protein; and d) optionally, carbohydrates such as lactose.

The composition may further comprise tricaproin.

The composition may comprise vegetable fats, such as palm oil, coconut oil, palm kernel oil, rapeseed oil, linseed oil, sunflower oil, MCT oil, and the like, and/or may comprise animal fats, such as lard, tallow, and/or insect fats.

The protein may comprise or consist of milk protein. The milk protein may comprise or consist of casein, and/or may comprise one or more of β-lactoglobulin, α-lactalbumin, blood serum albumin, immunoglobulins, lactoferrin, and transferrin.

The carbohydrates may comprise sugars such as lactose.

The present disclosure also provides a kit of parts, said kit comprising: a) a fat composition as taught herein; and b) a second composition comprising: i) 25-60 wt % fats such as vegetable and/or animal fats; ii) 18-75 wt % protein, e.g., milk protein and optionally vegetable protein; and iii) optionally, carbohydrates such as lactose, wherein preferably said second composition does not comprise tributyrin.

The present disclosure further provides a composition comprising: 15-35 wt %, preferably 15-30 wt %, based on dry matter content, fats; 18-28 wt %, based on dry matter content, protein; 30-55 wt %, preferably 35-55 wt %, based on dry matter content, sugars, said sugars preferably comprising lactose; said composition comprising at least 0.7 wt % based on dry matter content tributyrin.

Said composition may be in powder form or in aqueous form after reconstitution of a powder in an aqueous solution, e.g., in water.

Said composition may comprise less than about 5 wt % based on dry matter content starch and/or less than about 10 wt % based on dry matter content ash.

Said composition may comprise vegetable fats, such as palm oil, coconut oil, palm kernel oil, rapeseed oil, linseed oil, sunflower oil, MCT oil, or the like.

Said composition may comprise animal fats such as lard, tallow, or insect fats.

In an embodiment, the milk protein comprises or consists of casein, and/or the milk protein may comprise whey proteins such as β-lactoglobulin, α-lactalbumin, blood serum albumin, immunoglobulins, lactoferrin, and/or transferrin.

The composition may comprise lactose.

The present disclosure also teaches a method of preparing a composition as taught herein, said method comprising the step of mixing the fat composition taught herein or the kit of parts taught herein with milk protein and sugars to obtain said composition.

The present disclosure further provides a method of feeding a young mammal, preferably a young farming mammal, more preferably a calf, said method comprising the step of feeding to or administering to said young mammal a composition as taught herein.

Also, the present disclosure provides a method of increasing pre-weaning body weight gain, weaning body weight gain, post-weaning body weight gain, pre-weaning average daily gain, weaning average daily gain, post-weaning average daily gain, milk replacer intake, starter feed intake, dry matter intake, and/or a decreased energy conversion ratio, in a young mammal, preferably a young farming mammal, such as a calf, said method comprising the step of administering to said young mammal a composition as taught herein.

The present disclosure also provides a method of reducing diarrhea incidence and/or severity and/or respiratory disease incidence and/or severity in a young mammal, preferably a young farming mammal, such as a calf, of reducing the number of young farming mammals such as calves receiving delayed vaccination, and/or of reducing the number of young farming mammals such as calves receiving therapeutical interventions, said method comprising the step of administering to said young mammal a composition as taught herein.

Moreover, the present disclosure teaches a method of supporting or increasing gut development, supporting or increasing rumen development, increasing duodenal and/or ileal villi height, increasing duodenal villi width, increasing duodenal crypt width, increasing duodenal and/or ileal villi height to crypt depth ratio, and increasing duodenal and/or ileal muscle thickness, increasing ruminal polyp height, increasing ruminal polyp width, increasing ruminal papillae height, increasing ruminal papillae width, and/or increasing ruminal tunica submucosa, in a young mammal, preferably a young farming mammal, such as a calf, said method comprising the step of administering to said young mammal a composition as taught herein.

Finally, the present disclosure provides a method of increasing fat digestibility and/or of improving insulin sensitivity in a young mammal, preferably a young farming mammal, such as a calf, said method comprising the step of administering to said young mammal a composition as taught herein.

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references cited herein are incorporated herein in their entirety by reference.

The term “fats” as used herein refers to any compound comprising fatty acids. They include vegetable fats, animal fats, synthetic fats, fatty acid salts, esterified fatty acids, mono-, di-, triacylglycerols, and the like. The fats may have been processed, e.g., may have been synthetically made, e.g., like tributyrin and tricaproin, e.g., by esterification of glycerol and fatty acids, or may have undergone a certain degree of hydrogenation, e.g., partial hydrogenation or complete hydrogenation, interesterification, and the like.

The term ‘animal fats’ as used herein refers to lipids derived from animals. They may be liquid at room temperature, or solid. Although many animal parts and secretions may yield oil, in commercial practice, oil is extracted primarily from rendered tissue fats from livestock animals like pigs, chickens and cows. Dairy products yield animal fat and oil products such as butter.

The term “vegetable fats” as used herein refers to lipids obtained from plants, for example from seeds, from fruits, or from other parts of plants. Like animal fats, vegetable fats are mixtures of triglycerides.

The term “CMR” as used herein refers to calf milk replacer in the context of administration to a calf. However, it is interchangeable with the abbreviation “MR” for “milk replacer” and may refer to a milk replacer for any young mammal, preferably a young farming animal, such as a piglet, lamb, or goat kid.

Unless indicated otherwise herein, the weight percentages are on an as-is basis.

The term ‘about’, as used herein indicates a range of normal tolerance in the art, for example within 2 standard deviations of the mean. The term “about” can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”.

Reference to an element by the indefinite article “a” or “an” does not exclude the possibility that more than one of the elements is present, unless the context clearly requires that there be one and only one of the elements. The indefinite article “a” or “an” thus usually means “at least one”.

### DETAILED DESCRIPTION OF THE INVENTION

This patent application has been drafted into sections. However, these sections should not be read in isolation. Unless otherwise specified, each section is to be read in combination with the other sections. The various optional and preferred features can also be combined, even when taken from different parts of the specification. Likewise, all “aspects” and “embodiments” can be combined. No separation of embodiments is intended, unless explicitly stated.

### Fat Composition

In an aspect, the present disclosure provides a fat (concentrate) composition comprising at least 0.7 wt % tributyrin, 25-60 wt % fats; 18-75 wt %, or 20-75 wt %, such as 30-75 wt %, protein, preferably milk protein; and, optionally, carbohydrates such as lactose.

The fat composition taught herein may comprise 25-60 wt %, e.g., 25-55 wt %, or 25-50 wt %, such as 28-45 wt %, 30-42 wt %, or 30-40 wt %, fats.

The composition may further comprise vegetable protein.

The fats may comprise vegetable and/or animal fats, and/or may comprise synthetic fats.

In an embodiment, the fat comprises vegetable fats. Suitable vegetable fats include, without limitation, palm oil, coconut oil, palm kernel oil, rapeseed oil, linseed oil, sunflower oil, MCT oil, or the like. Suitable animal fats include, without limitation, tallow, lard, and insect fats. The fats may be partially or fully hydrogenated. The skilled person will be aware that any type of fats may be used.

In an embodiment, the fats comprise POP structural lipids. In another embodiment, the fat comprises both POP structural lipids and OPO structural lipids.

In an embodiment, said fats are not a combination of DHA algae oil powder, medium chain triglyceride, tributyrin, OPO structural lipid and lecithin.

In an embodiment, said fat (concentrate) composition comprises less than about 10 wt %, less than about 8 wt %, less than about 6 wt %, less than about 4 wt %, less than about 3 wt %, less than 2.5 wt %, less than 2 wt %, less than 1.5 wt %, less than 1 wt %, or less than 0.5 wt %, DHA algae oil powder. In an embodiment, said fat (concentrate) composition is essentially free or free from DHA algae oil powder. DHA algae oil powder can have a light fishy smell which can be unattractive to young farm animals such as calves and piglets can can hence negatively affect feed intake.

In an embodiment, said composition comprises less than about 2 wt %, preferably less than about 1.8 wt %, less than about 1.6 wt %, less than about 1.4 wt %, less than about 1.2 wt %, less than about 1 wt %, less than about 0.8 wt %, less than about 0.6 wt %, less than about 0.4 wt %, or less than about 0.2 wt % DHA. In an embodiment, said composition is essentially free of DHA or free from DHA. DHA can have a light fishy smell which can be unattractive to young farm animals such as calves and piglets and can hence negatively affect feed intake.

In an embodiment, said composition comprises less than about 2 wt %, preferably less than about 1.8 wt %, less than about 1.6 wt %, less than about 1.4 wt %, less than about 1.2 wt %, less than about 1 wt %, less than about 0.8 wt %, less than about 0.6 wt %, less than about 0.4 wt %, or less than about 0.2 wt %, medium-chain triglycerides comprising caprylic acid, capric acid, or a combination of the two. In an embodiment, said composition is essentially free of medium-chain triglycerides comprising caprylic acid, capric acid, or a combination of the two, or free from medium-chain triglycerides comprising caprylic acid, capric acid, or a combination of the two.

Said composition is preferably in solid form, preferably in powder form, at room temperature. Said fat composition may be obtainable by a method comprising the steps of heating the fats above their melting temperature to obtain fats in liquid form, mixing the fats with the protein, e.g. milk protein and, optionally, vegetable protein, and, optionally, carbohydrate such as lactose, to obtain a liquid mixture, and spray-drying said liquid mixture to obtain the fat composition, preferably in powder form, taught herein.

The fat composition taught herein preferably comprises less than about 20 wt %, more preferably less than 15 wt %, even more preferably less than 12 wt %, such as less than 10 wt % or less than 8 wt %, moisture.

The tributyrin may be present in the fat composition taught herein in any desired amount.

In an embodiment, the fat composition taught herein may comprise more than 0.7 wt %, more than 0.8 wt %, more than 0.9 wt %, more than 1 wt %, more than 1.25 wt %, more than 1.5 wt %, more than 1.75 wt %, preferably more than 2 wt %, or more than 2.5 wt %, yet more preferably more than 3 wt %, or more than 3.5 wt %, even more preferably more than 4 wt %, or more than 4.5 wt %, tributyrin. The maximum amount of tributyrin that can be incorporated in the fat composition taught herein will depend upon the type of fats present in the fat composition. So long as the melting temperature of the fats does not become too low, any amount of tributyrin may be included. Practically, the amount of tributyrin that may be incorporated into the fat composition taught herein may be less than 15 wt %, e.g., less than 14 wt %, less than 13 wt %, less than 12 wt %, less than 11 wt %, or less than 10 wt %.

The fat composition taught herein may further comprise tricaproin. The tricaproin may be present in the fat composition taught herein in any desired amount. In an embodiment, the fat composition taught herein may comprise more than 0.1 wt %, such as more than 0.2 wt %, more than 0.3 wt %, more than 0.4 wt %, more than 0.5 wt %, more than 0.6 wt %, more than 0.7 wt %, more than 0.8 wt %, more than 0.9 wt %, preferably more than 1 wt %, or more than 1.25 wt %, more preferably more than 1.5 wt %, or more than 1.75 wt %, even more preferably more than 2 wt %, more than 2.5 wt %, or more than 3 wt %, tricaproin. The maximum amount of tricaproin that can be incorporated in the fat composition taught herein will depend upon the type of fats present in the fat composition. So long as the melting temperature of the fats does not become too low, any amount of tricaproin may be included. Practically, the amount of tricaproin that may be incorporated into fat composition taught herein may be less than 15 wt %, e.g., less than 14 wt %, less than 13 wt %, less than 12 wt %, less than 11 wt %, or less than 10 wt %.

In an embodiment, the fats comprise vegetable fats. The vegetable fats may be selected from the group consisting of coconut oil, palm oil, linseed oil, rapeseed oil, soybean oil, grape seed oil, olive oil, rice bran oil, sunflower oil, and the like.

In an embodiment, the fats comprise animal fats selected from tallow, lard, and insect fats.

In an embodiment, the fats do not comprise milk fat.

The composition taught herein may further comprise 18-75, or 20-75 wt %, preferably 30-75 wt %, more preferably 20-65 wt %, more preferably 21-60 wt %, 22-55 wt %, 23-50 wt %, 24-45 wt %, or 25-40 wt %, protein. In a suitable embodiment, the fat composition taught herein comprises or consists of milk protein. In an embodiment, the protein comprises milk protein. In an embodiment, the protein further comprises vegetable protein. The milk protein preferably comprises casein or consists of casein. Alternatively, or additionally, the milk protein may comprise one or more of β-lactoglobulin, α-lactalbumin, blood serum albumin, immunoglobulins, lactoferrin, and transferrin. It may be advantageous to use by-products of the dairy industry to provide the milk protein, and, optionally, carbohydrates, to the fat composition taught herein. Suitable by-products of the dairy industry include, without limitation, skim milk, buttermilk, whey, or any product derived therefrom. As such products often also comprise lactose, a certain amount of lactose may be incorporated by using such dairy industry by-products.

In an embodiment, carbohydrates may be present in the fat composition taught herein. In an embodiment, the carbohydrates comprise lactose. In an embodiment, the fat composition comprises 20-40 wt %, lactose.

In a suitable embodiment, the fat composition comprises at least 0.7 wt % tributyrin, 25-45 wt % fats, 18-40 wt % protein, and 20-40 wt % lactose.

### Kit of Parts

The present disclosure also provides a kit of parts comprising a first and a second fat composition, said first fat composition being a fat composition as taught hereinabove, and said second fat composition being a fat composition as taught hereinabove albeit that said second fat composition is essentially free or entirely free of tributyrin. Said second fat composition may be a commercially available fat composition for the purpose of formulating milk replacers, in particular of formulating milk replacers for young farming mammals such as calves.

In an embodiment, said second fat composition comprises: a) 25-60 wt % fats, e.g. vegetable and/or animal fats; b) 18-75 wt %, e.g. 30-75 wt %, protein, e.g., milk protein and optionally vegetable protein; and c) optionally, carbohydrates such as lactose. Said second composition does not comprise tributyrin.

The second fat composition may comprise 25-60 wt %, e.g., 25-55 wt %, or 25-50 wt %, such as 28-45 wt %, 30-42 wt %, or 30-40 wt %, fat.

The second fat composition may comprise 18-75 wt %, or 20-75 wt %, preferably 30-75 wt %, more preferably 20-65 wt %, more preferably 21-60 wt %, 22-55 wt %, 23-50 wt %, 24-45 wt %, or 25-40 wt %, protein. The protein preferably comprises milk protein and may further comprise vegetable protein. In an embodiment, the second fat composition comprises 20-40 wt %, lactose.

In a suitable embodiment, the second fat composition comprises 25-45 wt % fats, 18-40 wt % protein, preferably milk protein and optionally vegetable protein, and 20-40 wt % lactose. The fats and proteins may be selected as described hereinabove in respect of the first fat composition.

The amount of the first fat composition and the second fat composition may depend on the tributyrin content and, optionally, tricaproin content, of the first fat composition, as well as the desired tributyrin and optionally tricaproin content of the milk replacer that is to be formulated. For example, if the first fat composition comprises 6 wt % tributyrin, and the desired tributyrin content of the milk replacer is 1 wt %, it may be advantageous to use 1 part of the first fat composition and 5 parts of the second fat composition. Alternatively, if the desired tributyrin content of the milk replacer is 0.5 wt %, it may be advantageous to use 1 part of the first fat composition and 11 parts of the second fat composition.

In this way, it is possible to tailor the tributyrin content and optionally the tricaproin content of the milk replacer to any desired amount. Moreover, it is possible to produce a limited number of batches of fat composition comprising tributyrin and optionally tricaproin, and combine them with cheaper and/or commercially available fat compositions.

### Milk Replacer Composition

In another aspect, the present disclosure teaches a (milk replacer) composition comprising:
i) 15-35 wt %, based on dry matter content, fat;
ii) 18-28 wt % based on dry matter content protein;
iii) 30-55 wt %, preferably 35-55 wt % based on dry matter content sugars, said sugars preferably comprising lactose,
said composition comprising at least 0.7 wt % tributyrin.

The composition may be a powder suitable for reconstitution into an aqueous solution or water to form a milk replacer. Alternatively, the composition may be a liquid milk replacer composition.

In an embodiment, said composition further comprises tricaproin.

Said composition is suitable to be used as a milk replacer for non-human young mammals, preferably young farming animals, such as calves, lambs, goat kids or piglets.

Calf milk replacers (CMRs) or milk replacers (MRs) provide a convenient way to feed non-human young mammals, such as young farming mammals, e.g., pre-ruminant calves. They can be stored long term as powder and mixed with water just prior to feeding. Young farming mammals such as calves can then be milk reared anywhere and at any time without having to source liquid whole milk. Thus, the composition taught herein may be in powder form, or may be in aqueous form after reconstitution of the powder in an aqueous solution such as water. The commercial product is usually a powder that is suitable for reconstitution in water on-farm.

Milk replacers may be formulated from by-products of dairy processing, together with animal and/or vegetable fats plus added vitamins and minerals The by-product of butter making is skim milk, which consists mainly of lactose and all the milk proteins. However, it has only half the energy value of whole milk. Whey, the by-product of cheese making, consists only of lactose, albumin and globulin, and is even lower in nutritive value. When these dairy processing by-products are used as the basis of milk replacers, additional fats are required.

The composition taught herein may comprise 15-35 wt %, e.g., 15-32 wt %, or 15-30 wt %, such as 15-28 wt %, 15-27 wt %, or 15-26 wt %, preferably 16-24 wt %, more preferably 17-23 wt %, even more preferably 18-22 wt %, based on dry matter content, fat. Said fat may comprise animal fats and/or vegetable fats. In an embodiment, the fat comprises vegetable fats. Suitable vegetable fats include, without limitation, palm oil, coconut oil, palm kernal oil, rapeseed oil, linseed oil, sunflower oil, MCT oil, or the like. Suitable animal fats include, without limitation, tallow, lard, and insect fats.

The composition taught herein may further comprise 18-28 wt %, preferably 19-27 wt %, more preferably 20-26 wt %, or 20-25 wt %, based on dry matter content, protein. Said protein preferably comprises proteins of milk origin (“milk proteins”), such as those from whey, skim milk and buttermilk powders. Spray-dried milk powder or whey powder are the preferred source of powder for milk replacers. In an embodiment, the milk protein comprises or consists of casein. In an embodiment, the milk protein comprises one or more of β-lactoglobulin, α-lactalbumin, blood serum albumin, immunoglobulins, lactoferrin, and transferrin.

The composition further comprises 30-55 wt %, preferably 35-55 wt %, based on dry matter content, sugars, said sugars preferably comprising or consisting of lactose, said sugars preferably being at least partially derived from dairy products by-processing.

In an embodiment, the composition comprises less than about 5 wt % based on dry matter content starch and/or less than about 10 wt % based on dry matter content ash.

In an embodiment, the fat comprises POP structural lipids. In another embodiment, the fat comprises both POP structural lipids and OPO structural lipids.

In an embodiment, said fats are not a combination of DHA algae oil powder, medium chain triglyceride, tributyrin, OPO structural lipid and lecithin.

In an embodiment, said fat (concentrate) composition comprises less than about 10 wt %, less than about 8 wt %, less than about 6 wt %, less than about 4 wt %, less than about 3 wt %, less than 2.5 wt %, less than 2 wt %, less than 1.5 wt %, less than 1 wt %, or less than 0.5 wt %, DHA algae oil powder. In an embodiment, said fat (concentrate) composition is essentially free or free from DHA algae oil powder. DHA algae oil powder can have a light fishy smell which can be unattractive to young farm animals such as calves and piglets can can hence negatively affect feed intake.

In an embodiment, said composition comprises less than about 2 wt %, preferably less than about 1.8 wt %, less than about 1.6 wt %, less than about 1.4 wt %, less than about 1.2 wt %, less than about 1 wt %, less than about 0.8 wt %, less than about 0.6 wt %, less than about 0.4 wt %, or less than about 0.2 wt % DHA. In an embodiment, said composition is essentially free of DHA or free from DHA. DHA can have a light fishy smell which can be unattractive to young farm animals such as calves and piglets and can hence negatively affect feed intake.

In an embodiment, said composition comprises less than about 2 wt %, preferably less than about 1.8 wt %, less than about 1.6 wt %, less than about 1.4 wt %, less than about 1.2 wt %, less than about 1 wt %, less than about 0.8 wt %, less than about 0.6 wt %, less than about 0.4 wt %, or less than about 0.2 wt %, medium-chain triglycerides comprising caprylic acid, capric acid, or a combination of the two. In an embodiment, said composition is essentially free of medium-chain triglycerides comprising caprylic acid, capric acid, or a combination of the two, or free from medium-chain triglycerides comprising caprylic acid, capric acid, or a combination of the two.

In an embodiment, the composition comprises an antioxidant to reduce the deterioration of fats during storage.

The composition taught herein may comprise tributyrin and tricaproin.

In an embodiment, the composition comprises at least 0.7 wt %, at least 0.8 wt % or at least 0.9 wt %, based on dry matter content of the composition, tributyrin.

In an embodiment, the composition comprises at least 0.1 wt %, such as at least 0.2 wt %, at least 0.3 wt %, at least 0.4 wt %, at least 0.5 wt %, or at least 0.6 wt %, based on dry matter content of the composition, tricaproin.

In a suitable embodiment, the composition comprises at least 0.7 wt %, at least 0.8 wt % or at least 0.9 wt %, based on dry matter content of the composition, tributyrin, and at least 0.1 wt %, such as at least 0.2 wt %, at least 0.3 wt %, at least 0.4 wt %, at least 0.5 wt %, or at least 0.6 wt %, based on dry matter content of the composition, tricaproin.

The tributyrin and if present, tricaproin, is/are part of the fat content of the composition. In a suitable embodiment, the tributyrin and optionally tricaproin are incorporated into the composition taught herein as part of the fat concentrate, e.g., a fat concentrate as taught herein.

Hence, in an embodiment, the composition taught herein comprises a fat concentrate as taught herein.

Particularly when in powder form, long-term storage of CMR or MR powders may be important. Hence, they may be packaged properly to keep out air and moisture. They may be vacuum sealed in a plastic bag then enclosed in a light-proof bag.

The composition taught herein may be provided with written instructions on how to reconstitute the composition in powder form in an aqueous solution, e.g., in water, to obtain the composition taught herein in aqueous form. Additionally, the written instructions may comprise further information on amount of feeding and/or frequency of feeding. Hence, the composition taught herein may be used as a milk replacer for young mammals, preferably young farming mammals.

The present disclosure also provides a method of preparing a composition as taught herein, said method comprising the step of mixing the fat composition taught herein or the kit of parts as taught herein with protein and sugars to obtain said composition.

### Methods and Uses of the Products Taught Herein

The present disclosure provides a method of feeding a young non-human mammal, preferably a young farming mammal, said method comprising the step of administering to said young non-human mammal, preferably said young farming mammal, such as a calf, lamb, piglet or goat kid, a (milk replacer) composition as taught hereinabove.

The present disclosure is also concerned with a method of increasing pre-weaning body weight gain, weaning body weight gain, post-weaning body weight gain, pre-weaning average daily gain, weaning average daily gain, post-weaning average daily gain, milk replacer intake, starter feed intake, dry matter intake, and/or a decreased energy conversion ratio, in a young mammal, preferably a young farming mammal, such as a calf, said method comprising the step of administering to said young mammal a composition as taught herein.

Additionally, the present disclosure provides a method of reducing diarrhoea incidence and/or severity and/or respiratory disease incidence and/or severity in a young mammal, such as a young farming mammal, of reducing the number of young farming mammals such as calves receiving delayed vaccination, and/or reducing the number of young farming mammals such as calves receiving therapeutical interventions, said method comprising the step of administering to said young mammal a composition as taught herein.

Moreover, the present disclosure teaches a method of supporting or increasing gut development, supporting or increasing rumen development, increasing duodenal and/or ileal villi height, increasing duodenal villi width, increasing duodenal crypt width, increasing duodenal and/or ileal villi height to crypt depth ratio, and increasing duodenal and/or ileal muscle thickness, increasing ruminal polyp height, increasing ruminal polyp width, increasing ruminal papillae height, increasing ruminal papillae width, and/or increasing ruminal tunica submucosa, in a young mammal, preferably a young farming mammal, such as a calf, said method comprising the step of administering to said young mammal a composition as taught herein.

Further, the present disclosure provides a method of increasing fat digestibility and/or of improving insulin sensitivity in a young mammal, preferably a young farming mammal, said method comprising the step of administering to said young mammal a composition as taught herein.

The young mammal is preferably a young non-human mammal, preferably a young farming mammal, such as a young pre-ruminant mammal, e.g., a calf such as a dairy calf or a beef calf, lamb, or a goat kid, or a young monogastric mammal, such as a piglet.

The (milk replacer) composition taught herein may be administered during any period of time and in any amount deemed suitable by the skilled person for raising said young mammal, preferably said young farming mammal such as calf, piglet, lamb, or goat kid.

The present invention is further illustrated, but not limited, by the following examples.

From the above discussion and the examples, one skilled in the art can ascertain the essential characteristics of the present invention, and without departing from the teaching and scope thereof, can make various changes and modifications of the invention to adapt it to various usages and conditions. Thus, various modifications of the invention in addition to those shown and described herein will be apparent to those skilled in the art from the foregoing description.

Such modifications are also intended to fall within the scope of the appended claims.

### EXAMPLES

### Example 1

A total of 24 male Holstein-Friesian calves (46.5±4.06 kg BW (mean±SD)) were gathered from neighbouring commercial dairy farms at an age of 36 to 96 h. A standardized colostrum management protocol, which included three feedings of colostrum in the first 24 h: 3.0 to 4.0 L within the first 1 h after birth, followed by two feedings of 2.0 L, was applied at the dairy farm of origin. Calves were assigned to 1 of 8 blocks of 3 calves, based on arrival day and age at arrival and within a block, calves were randomly assigned to 1 of the 2 treatments: a standard milk replacer (MR), or a MR including tributyrin (TB) and tricaproin (TC). Calves were monitored for 84 d, with the experimental period divided into 3 rearing phases: pre-weaning (P1), weaning (P2), and post-weaning (P3). In P1, calves were fed ad libitum MR from arrival to 42 d after arrival. In P2, calves were gradually weaned from a maximum daily consumption of 10.0 L MR on 43 d to 2.0 L on 70 d after arrival. Finally, in P3, including 71 d until 84 d after arrival, calves were fully weaned and fed exclusively solid feeds; starter feed and straw.

### Treatments and Feeding

Treatments consisted of two different experimental diets (n=12 per treatment): 1) MR with 30% fat (DM basis), including a blend of 58% palm, 32% coconut, and 10% linseed oils (“CON”), and 2) MR of treatment 1, including TB (3.36 wt % of fats) and TC (2.49 wt % of fats) in the fat concentrate (“TB+TC”). TB and TC were included at 6 wt % and 4 wt % of the fat concentrate, respectively. The rest of the fats in the initial fat concentrate of the TB+TC treatment were proportionally reduced by 10%. All MR were composed of 62.5% skimmed milk powder, 29.5% vegetable oils, 6.0% sweet whey powder, and 2.0% premix (vitamins, minerals, and additives) with a casein to whey ratio of 3.85. The MR diets were fed from arrival onward until 70 d after arrival. Milk replacer concentration was 135.0 g/L to reflect commercial recommendations regarding MR with high fat (>23% DM) and to get closer to the percentage of solids present in WM.

Calves were housed indoors in group pens. In each pen, calves received their respective MR treatment by electronic recognition with ad libitum access via one automated milk dispenser with the capacity to deliver two different MR treatments, starter feed (via one automated feeder, chopped wheat straw via one automated feeder, and water via one automated bowl. Milk replacer intake was monitored daily to meet at least 80% of the calf's ME requirements, based on weekly BW. The meal size from arrival until 42 d was set at maximum 2.0 L every 30 minutes, from 43 d until 70 d every 1.5 h calves were entitled to 0.5 L, which was build up to 2.0 L over time (6 h interval). Starter feed, chopped wheat straw, and water were available ad libitum throughout the experimental period.

### Measurements

The automated feeding systems recorded feed and water intakes daily by means of electronic recognition. Body weights were measured by weighing calves at arrival and on a weekly basis thereafter. Days of weighing varied according to the day of arrival within the week to minimize age differences. General animal health was monitored daily by farm technicians and therapeutic interventions (number of calves treated at least once) for respiratory diseases, diarrhoea, and other diseases including ear and navel infections were recorded. Calves were treated according to standard operating procedures.

### Calculations

Metabolizable energy content and intake was calculated for MR and starter feed. Milk replacer ME values were calculated as ME (McaVkg)=[0.057×CP (%)+0.092×CFAT (%)+0.0395×lactose (%)]×0.93 (NRC (National Research Council), 2001). Metabolizable energy value for starter feed was calculated based on the DE (McaVkg) of the same starter feed used in a previous study at the same location (Amado et al., 2022). Subsequently, the ME content was calculated by applying the ME equation from NRC (2001) for calf starters. The ME (MJ/kg) for MR and starter feed was calculated by multiplying the ME (McaVkg) by 4.184 (NRC, 2001). Total ME intake was calculated by summing the ME intake from MR and starter feed. Metabolizable energy intake from straw was not considered. The ECR was calculated by dividing the total ME intake (MJ/kg) from MR and starter feed by the ADG (kg).

### Results

Results for growth performance are presented in Table 1. There was a treatment by time interaction for overall BW (P<0.01) showing higher BW for TB+TC compared to CON. When considering the different rearing phases, BW tended to be greater for TB+TC than for CON during P1 (P=0.07) and was greater during P2 (P=0.02) and P3 (P=0.02). Body weight and ADG were greater for TB+TC than for CON (P<0.01) throughout the whole experimental period. Differences in ADG were present only in P2, where ADG was 16% and 15%, respectively, greater in calves fed TB+TC than calves fed CON (P=0.03). The overall Energy Conversion Ratio (ECR) tended to be higher for CON than for TB+TC (P=0.08). Throughout individual rearing phases, the ECR of the different treatments was similar.

TABLE 1
Average performance, milk replacer (MR), solids, and water intakes (Least Squares
Means) during pre-weaning (P1), weaning (P2), and post-weaning (P3) measured in male
Holstein calves fed different milk replacers1 (n = 12 per treatment)

| Item | CON | TB+TC | SEM | T | W | T*W |
|---|---|---|---|---|---|---|
| BW (kg)⁴ | | | | | | |
| P1 - Pre-weaning | 65.4 | 67.9 | 1.25 | 0.07 | <0.01 | 0.25 |
| P2 - Weaning | 94.5 | 102.4 | 2.16 | 0.02 | <0.01 | 0.35 |
| P3 - Post-weaning | 121.7 | 133.4 | 3.11 | 0.02 | <0.01 | 0.44 |
| Overall | 84.5 | 90.3 | 1.37 | <0.01 | <0.01 | <0.01 |
| ADG (g/d)⁴ | | | | | | |
| P1 - Pre-weaning | 812 | 933 | 53.7 | 0.15 | <0.01 | 0.88 |
| P2 - Weaning | 901 | 1076 | 55.7 | 0.03 | <0.01 | 0.82 |
| P3 - Post-weaning | 1438 | 1655 | 88.5 | 0.22 | 0.62 | 0.77 |
| Overall | 948 | 1101 | 35.0 | <0.01 | <0.01 | 0.99 |
| MR intake (L/d) | | | | | | |
| P1 - Pre-weaning | 7.75 | 8.32 | 0.396 | 0.37 | <0.01 | 0.48 |
| P2 - Weaning | 5.48 | 5.66 | 0.737 | 0.07 | <0.01 | 0.19 |
| P3 - Post-weaning | — | — | — | — | — | — |
| Overall | 6.84 | 7.26 | 0.240 | 0.19 | <0.01 | 0.79 |
| Starter feed intake (kg/d) | | | | | | |
| P1 - Pre-weaning | 0.04 | 0.03 | 0.008 | 0.71 | <0.01 | 0.06 |
| P2 - Weaning | 0.75 | 0.97 | 0.072 | 0.04 | <0.01 | 0.09 |
| P3 - Post-weaning | 3.32 | 3.71 | 0.162 | 0.13 | <0.01 | 0.53 |
| Overall | 0.82 | 0.96 | 0.044 | 0.03 | <0.01 | 0.07 |
| Straw intake (g/d) | | | | | | |
| P1 - Pre-weaning | 8 | 9 | 1.4 | 0.28 | <0.01 | <0.01 |
| P2 - Weaning | 138 | 141 | 13.6 | 0.18 | <0.01 | 0.64 |
| P3 - Post-weaning | 246 | 250 | 27.1 | 0.29 | 0.01 | 0.32 |
| Overall | 91 | 93 | 7.5 | 0.08 | <0.01 | 0.31 |
| Water intake (L/d) | | | | | | |
| P1 - Pre-weaning | 0.53 | 0.44 | 0.097 | 0.47 | <0.01 | 0.83 |
| P2 - Weaning | 2.59 | 3.27 | 0.335 | 0.34 | <0.01 | 0.16 |
| P3 - Post-weaning | 11.2 | 13.7 | 0.81 | 0.08 | <0.01 | 0.84 |
| Overall | 2.99 | 3.59 | 0.222 | 0.10 | <0.01 | 0.01 |
| ME (MJ/d) | | | | | | |
| P1 - Pre-weaning | 24.9 | 26.6 | 1.23 | 0.16 | <0.01 | 0.43 |
| P2 - Weaning | 23.4 | 26.2 | 0.70 | <0.01 | <0.01 | 0.84 |
| P3 - Post-weaning | 32.0 | 35.8 | 1.56 | 0.13 | <0.01 | 0.53 |
| Overall | 25.6 | 28.0 | 0.74 | <0.01 | <0.01 | 0.74 |
| Energy conversion (MJ/ kg BW | | | | | | |
| gain) | | | | | | |
| P1 - Pre-weaning | 46.2 | 32.2 | 5.91 | 0.22 | 0.18 | 0.63 |
| P2 - Weaning | 31.7 | 26.7 | 1.70 | 0.13 | <0.01 | 0.50 |
| P3 - Post-weaning | 24.9 | 22.4 | 2.01 | 0.58 | 0.07 | 0.45 |
| Overall | 37.9 | 28.8 | 2.91 | 0.08 | <0.01 | 0.80 |

¹In the pre-weaning phase (P1), calves were fed ad libitum MR from arrival to 42 d after arrival. In P2, calves were gradually weaned from 43 d to 70 d after arrival. In P3, including 71 d until 84 d after arrival, calves were fully weaned and fed exclusively solid feeds. Overall is defined as 0 d until 84 d after arrival. Water, straw, and starter feed were available ad libitum.
²Treatments included: CON = MR with 30% fat (DM basis), including a blend of 32% palm, 59% coconut, and 10% linseed oils (CON; n = 12), TRI = CON MR, including TB (3.36% of FA) and TC (2.49% of FA) in the fat concentrate (TRI; n = 12). Treatments concentration is 135.0 g DM/L.
³T = effect of treatment; W = effect of week; T*W = interaction between treatment and week.
⁴Analyzed with arrival BW as covariate.

Moreover, it was found that the number of treatment administrations (%) for diarrhea was lower for calves receiving TB+TC CMR compared to calves receiving control CMR (13% vs 20%), and that the number of treatment administrations for respiratory disease was also lower for calves receiving TB+TC CMR compared to calves receiving control CMR (6% vs 13%).

There was a trend for higher fat digestibility in the TB+TC treatment compared to CON.

### Rumen and Small Intestine Histomorphology

Images of the rumen, duodenum and ileum were examined. Morphometry analysis for duodenum, rumen and ileum included villi height, villi width, crypt height, crypt width, polyps and muscularis layer thickness were measured The villi height was measured from the top of the villi until the villi-crypt junction. The villi width was measured perpendicularly at mid-villi height. The same applies for the polyp and papillae for measuring height and width. The crypt depth was measured from the villi-crypt junction until the muscularis mucosae. The crypt height was measured as close as possible at the villi-crypt junction. Muscle thickness measurement includes the muscularis externa, containing the circular and longitudinal muscle. The results are set forth in Table 2.

TABLE 2
Villi height, villi width, crypt height, crypt depth and
muscle thickness of the duodenum, ileum and rumen in male
Holstein Friesian calves fed different milk replacer treatments
twice daily from arrival till 35.4 d ± 0.3 d.

| Item | CON | TB + TC |
|---|---|---|
| Duodenum | | |
| Villi height | 366 | 379 |
| Villi width | 173 | 179 |
| Crypt depth | 750 | 704 |
| Crypt width | 61.9 | 65.5 |
| Ratio VH:CD | 0.50 | 0.54 |
| SA | 1.55 | 1.54 |
| Muscle thickness | 593 | 644 |
| Ileum | | |
| Villi height | 326 | 382 |
| Villi width | 141 | 141 |
| Crypt depth | 530 | 532 |
| Crypt width | 57.9 | 56.3 |
| Ratio VH:CD | 0.61 | 0.71 |
| SA* | 1.40 | 1.27 |
| Muscle Thickness | 424 | 467 |
| Rumen, ventral sac | | |
| Polyp height | 1037 | 1044 |
| Polyp width* | 679 | 802 |
| Papillae height | 313 | 406 |
| Papillae width* | 174 | 206 |
| Tunica sub-mucosa* | 337 | 385 |
| Tunica muscularis | 2132 | 2040 |

Abbreviations: VH, villi height; CD, crypt depth; SA, surface area.
Expressed in μm unless specified otherwise.
Treatments included 2 milk replacers (MR): a MR with vegetable oils without C4:0 and C6:0 (CON), and a MR with vegetable oils and inclusion of tributyrin and tricaproin (TB + TC) fed at 135 g/L.

Results for duodenum, ileum and rumen parameters are found in Table 2. Rumen development was improved considerably in calves receiving CMR comprising TB and TC compared to those receiving CON CMR. Moreover, ileal villi height was increased considerably in calves receiving CMR comprising TB and TC compared to those receiving CON CMR.

Insulin sensitivity was also found to be improved in calves receiving CMR comprising TB and TC compared to those receiving CON CMR.

### Example 2

Beef calves received standard CMR from day 0 to day 14, and were fed either CON CMR or CON CMR+1 wt % based on dry matter content tributyuin (“CMR+TB”) from day 14 onward. 15 calves were used for each treatment.

It was found that the number of calves for which vaccination needed to be delayed due to therapeutical interventions was decreased for calves receiving CMR+TB compared to CON CMR (7% delayed vaccination for CMR+TB vs. 33% delayed vaccination for CON CMR). Moreover, it was found that the percentage of calves receiving therapeutical interventions was decreased for calves receiving CMR+TB compared to CON CMR (76% for CMR+TB vs. 96% for CON CMR). Moreover, the percentage of calves having scours was lower for the CMR+TB calves compared to the CON CMR calves (32% vs 45%) and the CMR+TB calves could be treated for a shorter period of time compared to the CON CMR calves (3.5 days vs 6 days) indicating that the scours were less severe for CMR+TB calves compared CON CMR calves.

1. A composition comprising:
a) at least 0.7 wt % tributyrin;
b) 25-60 wt % fats;
c) 18-75 wt % protein; and
d) optionally, carbohydrates.

2. The composition according to claim 1, wherein the composition further comprises tricaproin.

3. The composition according to claim 2, wherein the fats comprise vegetable fats.

4. The composition according to claim 3, wherein the vegetable fats comprise palm oil, coconut oil, palm kernel oil, rapeseed oil, linseed oil, sunflower oil, or MCT oil.

5. The composition according to claim 1, wherein the protein comprises or consists of milk protein.

6. The composition according to claim 5, wherein the milk protein comprises one or more of casein, β-lactoglobulin, α-lactalbumin, blood serum albumin, immunoglobulins, lactoferrin, and transferrin.

7. The composition according to claim 1, wherein the carbohydrates comprise sugars.

8. A kit of parts comprising:
a) a composition according to claim 1; and
b) a second composition comprising:
i) 25-60 wt % fats;
ii) 18-75 wt % protein; and
iii) optionally, carbohydrates,
wherein the second composition does not comprise tributyrin.

9. A composition comprising:
i) 15-35 wt %, based on dry matter content, fats;
ii) 18-28 wt %, based on dry matter content, protein;
iii) 30-55 wt %, based on dry matter content, sugars;
wherein the composition comprises at least 0.7 wt %, based on dry matter content, tributyrin.

10. The composition according to claim 9, wherein the composition is in powder form or in aqueous form after reconstitution of a powder in an aqueous solution.

11. The composition according to claim 9, wherein the composition comprises, based on dry matter content, less than about 5 wt % starch and/or less than about 10 wt % ash.

12. The composition according to claim 1, wherein the fats comprise vegetable fats.

13. The composition according to claim 12, wherein the vegetable fats comprise palm oil, coconut oil, palm kernel oil, rapeseed oil, linseed oil, sunflower oil, or MCT oil.

14. The composition according to claim 9, wherein the protein comprises or consists of milk protein.

15. The composition according to claim 14, wherein the milk protein comprises one or more of casein, β-lactoglobulin, α-lactalbumin, blood serum albumin, immunoglobulins, lactoferrin, and transferrin.

16. The composition according to claim 9, wherein the sugars comprise or consist of lactose.

17. A method of feeding a young mammal, the method comprising the step of feeding to the young mammal a composition according to claim 9.

18. A method of preparing a composition according to claim 9, the method comprising the step of mixing a fat composition according to claim 1 or a kit of parts according to claim 8 with protein and sugars to obtain the composition.

19. A method of increasing pre-weaning body weight gain, weaning body weight gain, post-weaning body weight gain, pre-weaning average daily gain, weaning average daily gain, post-weaning average daily gain, milk replacer intake, starter feed intake, dry matter intake, and/or a decreased energy conversion ratio, in a young farming mammal, the method comprising the step of administering to the young mammal a composition according to claim 9.

20. A method of reducing diarrhea incidence and/or severity and/or respiratory disease incidence and/or severity in a young farming mammal, of reducing the number of young farming mammals receiving delayed vaccination, and/or reducing the number of young farming mammals receiving therapeutic interventions, the method comprising the step of administering to the young farming mammal a composition according to claim 9.

21. A method of supporting or increasing gut development, supporting or increasing rumen development, increasing duodenal and/or ileal villi height, increasing duodenal villi width, increasing duodenal crypt width, increasing duodenal and/or ileal villi height to crypt depth ratio, increasing duodenal and/or ileal muscle thickness, increasing ruminal polyp height, increasing ruminal polyp width, increasing ruminal papillae height, increasing ruminal papillae width, and/or increasing ruminal tunica submucosa, in a young farming mammal, the method comprising the step of administering to the young farming mammal a composition according to claim 9.

22. A method of increasing fat digestibility and/or of improving insulin sensitivity in a young farming mammal, the method comprising the step of administering to the young mammal a composition according to claim 9.

23. The method according to claim 17, wherein the young farming mammal is selected from a calf, piglet, lamb, and goat kid.
